(19) United States
(12) Patent Application Publication
(10) Pub. No.: US 2023/0035669 A1
(43) Pub. Date: Feb. 2, 2023

(54) Fluid Output Measurement and Analysis System

(71) Applicant: C. R. Bard, Inc., Franklin Lakes, NJ (US)

(72) Inventors: Vijaya Sundara Pandian Raja, Chennai (IN); Reema Chaudhary, Panchkula (IN); Neegam Singh, Chandigarh (IN)

(21) Appl. No.: 17/873,834

(22) Filed: Jul. 26, 2022

Related U.S. Application Data
(60) Provisional application No. 63/228,451, filed on Aug. 2, 2021.

(51) Int. Cl. G01F 23/00; A61B 5/20; A61B 10/00

(52) U.S. Cl. CPC G01F 23/0007; A61B 5/208; A61B 10/007

(57) ABSTRACT

Embodiments described herein are directed to an automatic fluid measurement system including a fluid collection system configured to drain a fluid from a patient, and a short wave infrared imaging system configured to image the collection container to provide a high contrast, high resolution image and determine a volume of fluid disposed therein. In an embodiment, the system can determine a fluid flow rate, or determine a presence or concentration of foreign particles within the collection container. One or more cameras can image different portions of the container, or can include different focal lengths. Images of the collection container can be communicated to remote computing devices for further analysis.

### PRIORITY

This application claims the benefit of priority to U.S. Provisional Application No. 63/228,451, filed Aug. 2, 2021, which is incorporated by reference in its entirety into this application.

### SUMMARY

Briefly summarized, embodiments disclosed herein are directed to fluid output measurement and analysis systems using short wave infrared (“SWIR”) modalities, and associated methods thereof. Current automatic fluid output measurement systems often rely on measuring a change in weight of the collection container to determine a change in fluid volume over time, and thereby a fluid flow rate. Such systems often require sturdy bases and large support frames to increase stability and improve accuracy in detecting changes in weight in low-flow situations. However, in a clinical setting, such systems can take up valuable floor space and can be cumbersome to lift and transport.

Embodiments disclosed herein are directed to fluid output measurement and analysis systems using short wave infrared (“SWIR”) cameras. Such systems can provide high accuracy of fluid flow data, while reducing the size and footprint of the overall system, improving mobility, and improving overall discretion of the device. Further, such systems can analyze samples of the fluid without breaching the closed fluid collection system.

Disclosed herein is an automatic fluid output measurement system including, a fluid output measurement device having a housing including a back plate supporting a short wave infrared (“SWIR”) camera, and a shield extending parallel to the back plate and supported in spaced apart relationship therefrom, a fluid collection system including a collection container disposed between the back plate and the shield, and an image processing device disposed within the housing and communicatively coupled to the SWIR camera, the image processing device configured to image the collection container using short wave infrared electromagnetic radiation to determine a volume of fluid disposed therein.

In some embodiments, the automatic fluid output measurement system further includes a SWIR light source configured to emit short wave infrared electromagnetic radiation onto the collection container.

In some embodiments, the automatic fluid output measurement system further includes a second camera communicatively coupled to the image processing device.

In some embodiments, the second camera is disposed at one or both of a same tilt angle and a same focal length as the SWIR camera.

In some embodiments, the second camera is disposed at one or both of a different tilt angle and a different focal length as the SWIR camera.

In some embodiments, the SWIR camera is further configured to image the collection container using one or more of SWIR, medium wave infrared radiation (“MWIR”), long wave infrared radiation (“LWIR”), visible light, ultraviolet light, and infrared radiation (“IR”).

In some embodiments, the collection container includes one or more graduated markings configured to provide a high contrast graduated marking relative to the collection container when imaged by the SWIR camera.

In some embodiments, the collection container is configured to be suspended from the housing.

In some embodiments, the housing includes a clamp configured to couple the housing to a supporting structure.

In some embodiments, the image processing device is communicatively coupled to an external computing device.

In some embodiments, the image processing device is configured to determine one or both of a presence of a foreign particle and a concentration of a foreign particle within the collection container.

In some embodiments, the collection container is in fluid communication with a Foley catheter.

Also disclosed is a method of measuring a fluid from a patient including, draining a fluid to a collection container, imaging the collection container using a first camera configured to detect short wave infrared electromagnetic radiation, and determining a fluid volume within the collection container.

In some embodiments, the method further includes determining a change in fluid volume over time to determine a fluid flow.

In some embodiments, the method further includes determining one or both of a presence of a foreign particle within the collection container and a concentration of a foreign particle within the collection container.

In some embodiments, the method further includes detecting a first image using the first camera, and a second image using a second camera.

In some embodiments, the first camera images one or both of a different portion of the collection container, a different angle of the collection container, and a different focal length, relative to the second camera.

In some embodiments, the camera is supported by a housing having a back plate and a shield extending parallel to the back plate and supported in a spaced apart relationship therefrom, the collection container disposed between the back plate and the shield.

In some embodiments, the method further includes suspending the housing from a supporting structure.

In some embodiments, the method further includes imaging a graduated marking under short wave infrared electromagnetic radiation and comparing a fluid level relative to the graduated marking to determine a fluid volume.

In some embodiments, the method further includes communicating the image of the collection container to an external computing device.

In some embodiments, draining a fluid further includes draining urine from a catheter to the collection container.

In some embodiments, the collection container includes one of a plastic, polymer, High Density Polyethylene (HDPE), Polyethylene Terephthalate (PET), Polypropylene (PP), Low Density Polyethylene (LDPE), or Polyvinyl Chloride (PVC).

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a fluid collection system in an exemplary environment of use, in accordance with embodiments disclosed herein.

FIG. 2A shows a perspective view of a fluid output measurement device, in accordance with embodiments disclosed herein.

FIG. 2B shows a perspective view of an automatic fluid output system including a fluid collection system and a fluid output measurement device, in accordance with embodiments disclosed herein.

FIG. 3A shows a side view of an automatic fluid output system, in accordance with embodiments disclosed herein.

FIG. 3B shows a side view of an automatic fluid output system, in accordance with embodiments disclosed herein.

FIG. 3C shows a side view of an automatic fluid output system, in accordance with embodiments disclosed herein.

FIG. 4A shows a perspective view of an automatic fluid output system viewed under visible light, in accordance with embodiments disclosed herein.

FIG. 4B shows a perspective view of an automatic fluid output system viewed under SWIR light, in accordance with embodiments disclosed herein.

FIG. 4C shows close up detail of exemplary foreign particles within the fluid collection system when viewed under visible light, in accordance with embodiments disclosed herein.

FIG. 4D shows close up detail of exemplary foreign particles within the fluid collection system when viewed under SWIR light, in accordance with embodiments disclosed herein.

FIG. 5 shows a schematic view of an image processing device of an automatic fluid output system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

### Terminology

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Singular forms of “a,” “an,” and “the” include plural references unless the context clearly dictates otherwise.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage. In an embodiment, the logic described herein may rely on heuristics, machine learning, artificial intelligence (A.I.), neural networks, or other data processing techniques or similar artificial intelligence schema to perform the described functionality.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

A “message” generally refers to information transmitted in one or more electrical signals that collectively represent electrically stored data in a prescribed format. Each message may be in the form of one or more packets, frames, HTTP-based transmissions, or any other series of bits having the prescribed format.

As used herein “wireless” communication modalities can include but not limited to WiFi, Bluetooth, Near Field Communications (NFC), cellular Global System for Mobile Communication (“GSM”), electromagnetic (EM), radio frequency (RF), combinations thereof, or the like.

The term “computerized” generally represents that any corresponding operations are conducted by hardware in combination with software and/or firmware.

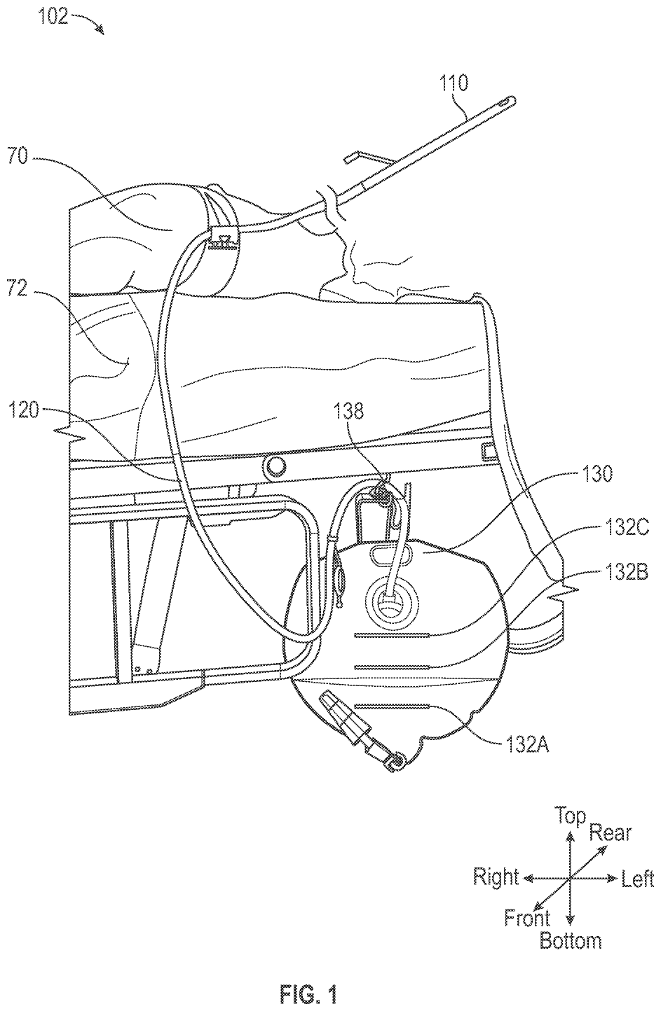

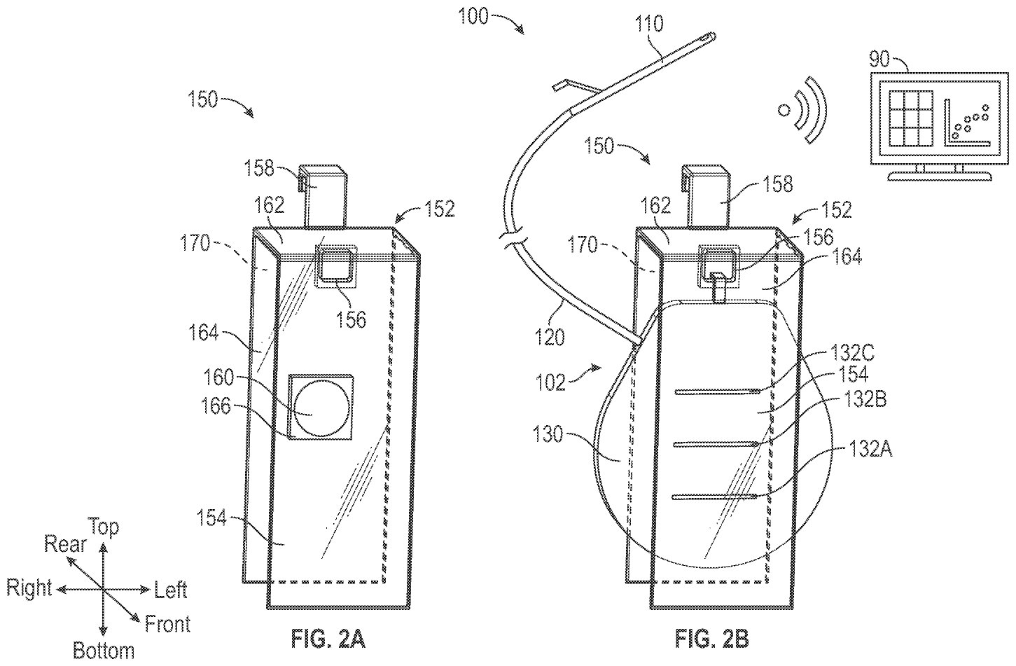

Labels such as “left,” “right,” “upper”, “lower,” “top,” “bottom,” “front,” “back,” and the like are used for convenience and are not intended to imply, for example, any particular fixed location, orientation, or direction. Instead, such labels are used to reflect, for example, relative location, orientation, or directions. To assist in the description of embodiments described herein, the “top,” “bottom,” “left,” “right,” “front” and “back” directions are in reference to the orientation of the device as shown in FIGS. 1-2A. A vertical axis extends between a top direction and a bottom direction. A lateral axis extends horizontally between a left direction and a right direction, substantially normal to the vertical axis. A transverse axis extends horizontally between a front direction and a back direction, substantially normal to both the vertical and lateral axes. A horizontal plane is defined by the lateral and transverse axes. A median plane is defined by the vertical and transverse axes. A frontal plane is defined by the vertical and lateral axes.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

FIG. 1 shows an exemplary fluid collection fluid collection system 102 in an exemplary environment of use and configured for draining a fluid from a cavity of a patient 70. The fluid collection system 102 can generally include a catheter 110, or similar device, and a drainage tube 120 in fluid communication with a collection container 130. In an embodiment, the fluid collection system 102 including the catheter 110, drainage tube 120, and collection container 130 can form a “closed” fluid collection system 102 to mitigate the introduction of pathogens or similar catheter-associated urinary tract infections (“CAUTI”) causing agents.

Fluid can flow from the catheter 110 through the drainage tube 120 and into the collection container 130. In an embodiment, the fluid collection system 102 can be a passive system, e.g. a gravity fed system. In an embodiment, the fluid collection system 102 can be an active system and further include one or more pumping mechanisms, or the like, configured to urge the fluid proximally through the fluid collection system 102 from the catheter 110 to the container 130. In an embodiment, the catheter 110 can be an internal catheter or an external catheter. Exemplary catheters can include external urinary catheter, internal urinary catheter, Foley catheter, balloon catheter, peritoneal catheters, or the like. Exemplary fluids collected can include urine, blood, peritoneal fluid, interstitial fluid, pus or the like. In an embodiment, the catheter 110 can be a Foley catheter configured to drain a fluid, e.g. urine, from a bladder of a patient 70.

In an embodiment, the collection container 130 can be a flexible bag, a rigid container, combinations thereof, or any similar suitable container configured to collect a fluid from a patient. In an embodiment, the collection container 130 can be formed of flexible or rigid, transparent, or translucent material such as a plastic, polymer, High Density Polyethylene (HDPE), Polyethylene Terephthalate (PET), Polypropylene (PP), Low Density Polyethylene (LDPE), Polyvinyl Chloride (PVC), Polystyrene (PS), Polycarbonates (PC), Acrylonitrile-Butadiene-Styrene (ABS) combinations thereof, or the like.

As shown in FIG. 1, the collection container 130 can include a hook 138 configured to releasably engage a supporting structure 72 (e.g. a patient bed, gurney, wheel chair, or the like) and suspend the collection container 130 therefrom. Advantageously, suspending the collection container 130 from a patient bed, gurney, wheel chair, or the like can improve portability of the fluid collection system 102, or the automatic fluid output system 100 as a whole, relative to suspending the fluid collection system 102 from a stand, or separate structure which can also take up additional floor space. Further, suspending the fluid collection system 102 below a bed, gurney, wheel chair, or the like can provide a discrete storage location, improving patient comfort.

FIGS. 2A-2B show further details of an automatic fluid output system (“system”) 100 including a fluid collection system 102 and a fluid (e.g. urine) output measurement device (“UO device”) 150. FIG. 2A shows the UO device 150 alone. FIG. 2B shows the system 100 including the fluid collection system 102 coupled with the UO device 150 and, optionally, an external computing system 90 communicatively coupled thereto.

The UO device 150 can generally include a housing 152 having a back plate 164 supporting a short wave infrared (“SWIR”) camera 160, a shield 154 supported in a spaced apart relationship from the back plate 164, a bag-hanger 156, and a clamp 158. To note, the shield 154 is shown in wire frame for ease of illustration. The back plate 164 of the housing 152 can extend over a frontal plane and can define a top edge, a bottom edge, a right side edge and a left side edge. The top plate 162 can extend forwards from the top edge of the back plate 164, along a transverse plane, perpendicular to the plane of the back plate 164 and can support the shield 154 in a spaced apart relationship from the back plate 164. The shield 154 can extend downwards from a front edge of the top plate 162, perpendicular to the top plate 162 and substantially parallel to the back plate 164. The top plate 162 can be configured to support the shield 154 in a spaced apart relationship from the back plate 164. It will be appreciated however, that the terms “back plate,” “top plate,” and “shield” are not intended to be limiting and that other configurations and orientations of back plate 164, top plate 162, and shield 154 are contemplated.

The UO device 150 can further include a bag hanger 156 coupled to the top plate 162, or coupled to one of the back plate 164 or the shield 154 and disposed proximate the top of the device 150. For example, the bag hanger 156 can be coupled to a front surface of the housing 152 proximate the top edge thereof, a rear surface of the shield 154 proximate the top edge thereof, or can be coupled to a bottom surface of the top plate 162. However, it will be appreciated that other configurations of bag hanger 156 are also contemplated. In an embodiment, the bag hanger 156 can include a hook, a loop, clip, clamp, or a similar structure configured to releasably engage the hook 138 of the collection container 130 and suspend the collection container 130 therefrom. In an embodiment, the UO device 150 can further include a clamp 158 and include a hook, loop, clip, magnet, chord, zip tie, VELCRO™ loop, or similar structure configured to releasably secure the UO device 150, and collection container 130 coupled there to, to a supporting structure 72 such as a patient bed, gurney, wheel chair, or the like, as described herein.

In an embodiment, the UO device 150 can include a short wave infrared (“SWIR”) modality camera 160 and a SWIR modality light source 166. The SWIR camera 160 can be configured to detect electromagnetic radiation (e.g. light) within the SWIR frequency range. It will be appreciated that the camera 160 can also be configured to detect other ranges of electromagnetic radiation including infrared light, near infrared light, optical light, ultraviolet light, combinations thereof, or the like. In an embodiment, the SWIR light source 166 can include one or more light bulbs, LED's, combinations thereof, or the like, configured to emit short wave infrared (SWIR) light. In an embodiment, the SWIR light source 166 can be disposed annularly about the SWIR camera 160, however, other configurations of light source 166 are contemplated to fall within the scope of the present invention.

In an embodiment, the SWIR light source 166 can be configured to emit SWIR light onto the collection container 130, or a portion thereof. The SWIR camera 160 can be configured to detect short wave infrared (SWIR) light. In an embodiment, the SWIR camera 160 can detect SWIR light reflected off of the container 130 to provide an image of the container 130.

In an embodiment, the SWIR light source 166 can disposed on a front side of the back plate 164 and can illuminate a rear side of the collection container 130. In an embodiment, the SWIR light source 166 can be disposed on a rear side of the shield 154 and can illuminate a front side of the collection container 130. In an embodiment, the SWIR camera 160 can be disposed on the back plate 164 and image a rear side of the collection container 130. In an embodiment, the SWIR camera 160 can be disposed on the shield 154 and image a front side of the collection container 130. These and other configurations of light source 166 and camera 160 are contemplated to fall within the scope of the present invention.

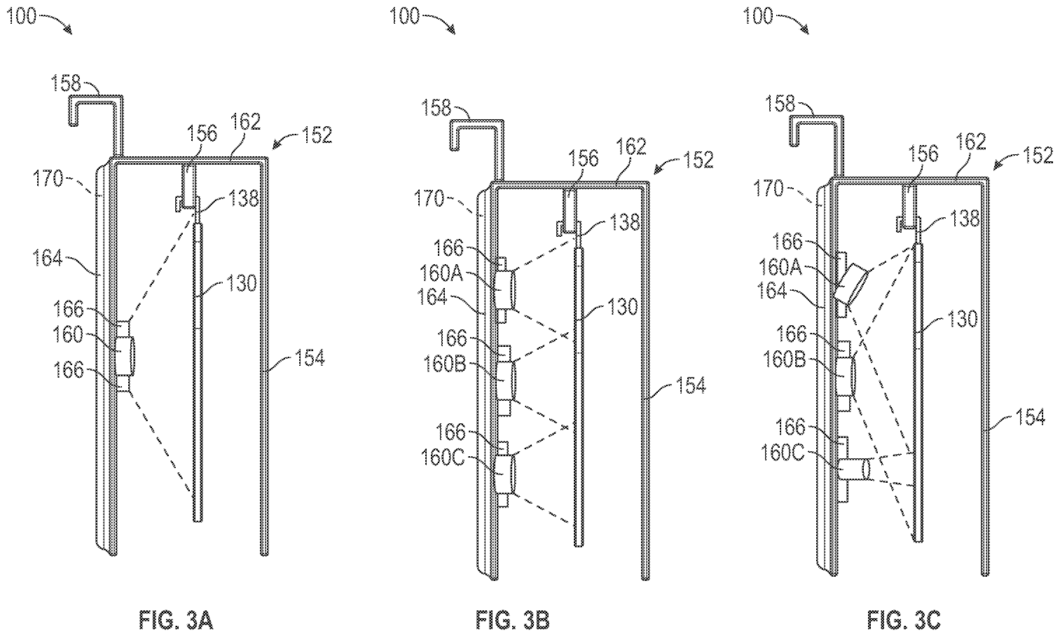

In an embodiment, as shown in FIGS. 3A-3C, the focal length of the camera 160 lens can be adjustable and tiltable to cover a desired area of the collection container 130. In an embodiment, the UO device 150 can include one or more SWIR camera's 160 disposed at different positions on the housing 150 (e.g. a first camera 160A, second camera 160B, third camera 160C). Images from each of the cameras 160 can then be combined and/or analyzed to ensure the collection container 130, or portion thereof, is imaged correctly. In an embodiment, as shown in FIG. 3B each of the cameras 160A-160C can have the same tilt angle or focal length to cover the collection container 130 evenly. In an embodiment, as shown in FIG. 3C, one or more cameras 160A-160C can have a different tilt angle or focal length to gather images from different areas of the collection container 130 or at different resolutions as required to fully image and analyze the fluid disposed therein, as described in more detail herein.

As used herein, short wave infrared (SWIR) light can include electromagnetic (EM) radiation having a wave length of between 900 nm (0.9 μm) and 1700 nm (1.7 μm). In an embodiment, one of the light source 166 and/or the camera 160 can be configured to emit and/or detect infrared (IR) electromagnetic radiation having a wavelength of between 750 nm (0.75 μm) and 12000 nm (12.0 μm). In an embodiment, one of the light source 166 and/or the camera 160 can be configured to emit and/or detect visible light electromagnetic radiation having a wavelength of between 400 nm (0.4 μm) and 750 nm (0.75 μm). In an embodiment, one of the light source 166 or the camera 160 can be configured to emit or detect one of SWIR, IR, or visible light EM, or combinations thereof. In an embodiment, the UO device 150 can include two or more light sources 166 or two or more cameras 160, each configured to emit or detect one or more of SWIR, IR, or visible light EM, or combinations thereof.

Advantageously, imaging the container 130 using SWIR light provides a strong contrast image, or “sharp” image relative to thermal imaging, which relies on infrared (IR), or longer wave infrared radiation, (e.g. medium wave infrared radiation (“MWIR”), or long wave infrared radiation (“LWIR”)). Advantageously, the SWIR camera can detect electromagnetic radiation in a similar manner to visible light. SWIR photons are reflected or absorbed by an object, providing the strong contrast needed for higher resolution imaging. Further, SWIR light is reflected or absorbed differently from visible light electromagnetic radiation providing high contrast between materials or fluids that would otherwise appear uniform when imaged under visible light electromagnetic radiation. By contrast, thermal images from infrared cameras rely on radiated photons and provide relatively lower resolution images.

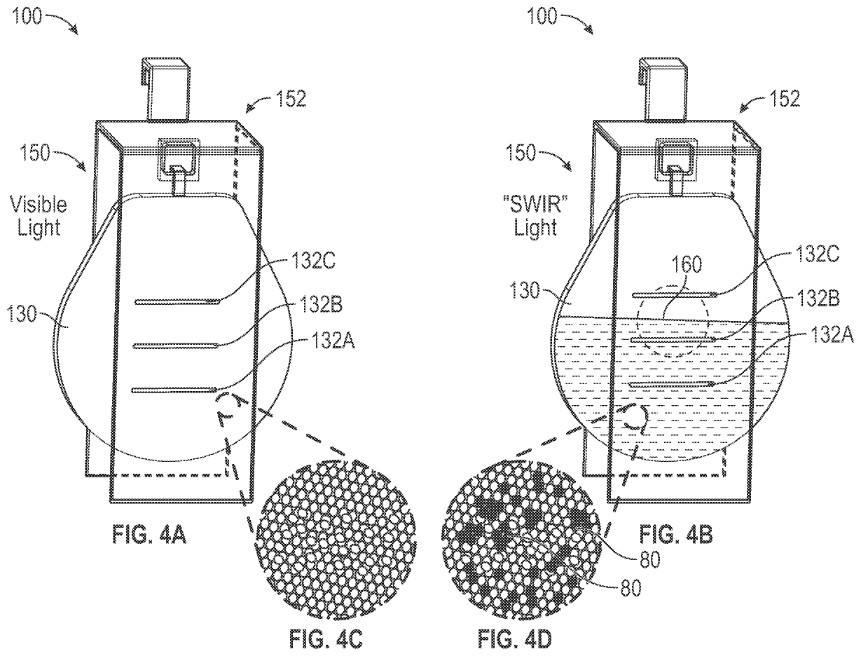

FIG. 4A shows a schematic view of a collection container 130 including a fluid disposed therein, imaged under visible light. FIG. 4B shows a schematic view of the same collection container 130 including the same volume of fluid disposed therein, and imaged under SWIR light. Advantageously, the SWIR camera 160 using SWIR light can image the collection container 130 and provide a high contrast, high resolution image of a fluid level disposed within the container 130. Advantageously, the shield 154 can be configured to provide a uniform background to the SWIR image of the collection container 130 to facilitate providing the high contrast, high resolution image of the fluid level within the collection container 130. In an embodiment, the collection container 130 can include one or more graduated markings 132 disposed on a surface thereof. The graduated markings 132 can be printed, engraved, embossed, molded, or combinations thereof. In an embodiment, the graduated markings 132 can be configured to provide a high contrast graduated line when imaged under SWIR light.

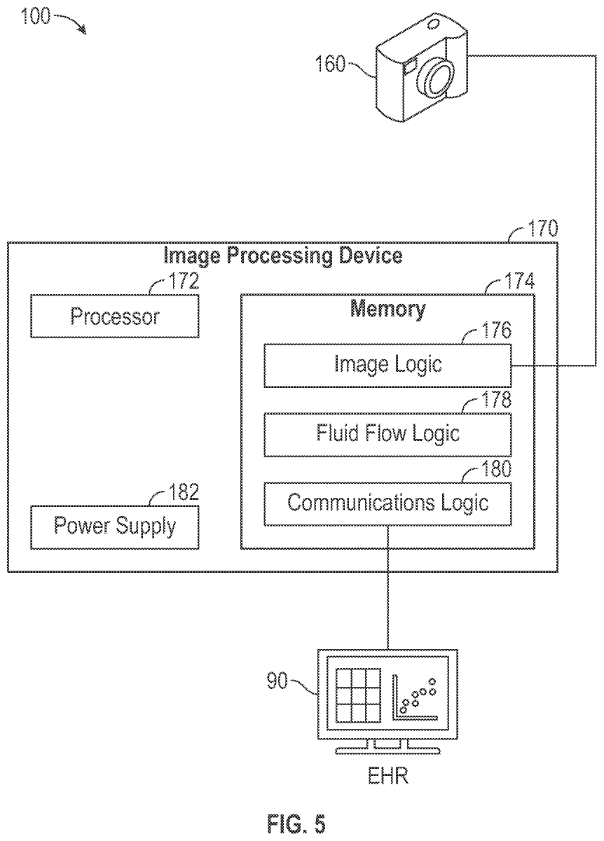

FIG. 5 shows a schematic view of the automatic fluid output system 100 including an image processing device 170 disposed within the housing 152 of the UO device 150. In an embodiment, the image processing device 170 can include one or more hardware, software, or electrical components, such as processors 172, non-transitory or transitory memory 174, image logic 176, fluid flow logic 178, communications logic 180, power source 182, combinations thereof, or the like, configured to receive image information from the SWIR camera 160. In an embodiment, the power source 182 can be a mains power supply, battery power supply, combinations thereof, or similar suitable power supply.

In an embodiment, the image logic 176 can receive information from the SWIR camera 160 and can determine an image of the collection container 130 including a fluid level disposed therein. In an embodiment, the fluid flow logic 178 can receive image information from the image logic 176 and can determine a volume of fluid disposed within the collection container 130. As noted, the collection container 130 can include one or more graduated markings 132 disposed on a surface thereof. In an embodiment, the fluid flow logic 178 can measure a fluid level relative to one or more graduated markings 132 and can determine a fluid volume disposed within the collection container 130. In an embodiment, the one or more graduated markings 132 can be associated with qualitative measurements, e.g. low 132A, middle 132B, and high 132C; empty 132A, middle 132B, and full 132C). In an embodiment, the one or more graduated markings 132 can be associated with quantitative measurements, e.g. 500 ml 132A, 1300 ml 132B, and 2500 ml 132C. These and other combinations of qualitative and/or quantitative graduated markings are contemplated to fall within the scope of the present invention. In an embodiment, the fluid flow logic 178 can determine a change in fluid level and a therefore a change in fluid volume over time. As such, the fluid flow logic 178 can determine a fluid flow rate.

In an embodiment, as shown in FIGS. 4C-4D, the image logic 176 can be configured to analyze the image of the collection container 130 to determine the presence or absence of particular target foreign particles 80. Exemplary foreign particles 80 can include solutes, molecules, sugars, carbohydrates, urea, minerals, calcium oxylates, cells, red blood cells, puss cells, bacteria, pathogens, combinations thereof, or the like. For example, as shown in FIG. 4C, when imaging the fluid disposed within the collection container 130 under visible light, the fluid can appear substantially uniform. By contrast, when imaging the same fluid within the container 130 under SWIR light, certain foreign particles 80 can reflect SWIR light differently from the surrounding fluid. As such, the image logic 176 can provide a high contrast, high resolution image of these foreign particles 80 which can be analyzed to determine the presence/absence or concentration of these foreign particles 80 within the fluid.

In an embodiment, the image processing device 170 can communicate one of the image information or fluid flow information either by wired or wireless communication, to one or more external computing devices 90, e.g. a laptop, computer, handheld device, tablet, mobile device, smart phone, network, server, hospital network, electronic health record (EHR) system, combinations thereof or the like. In an embodiment, wireless communication can include WiFi, Bluetooth, NFC, or similar modality. However it will be appreciated that other modalities are also contemplated.

Advantageously, the automatic fluid output system 100 including the UO device 150 and image processing device 170 can remotely monitor a fluid volume and flow rate of fluid within the fluid collection system 102 without a clinician having to directly observe the collection container 130. Flow rate measurements can be taken accurately and at regular time intervals without requiring a user (e.g. nursing staff member) to measure and record fluid flow data, reducing the work load demands of the clinicians.

Advantageously, the UO device 150 can measure the fluid volume or flow rate within a closed fluid collection system 102, without having to access the collection fluid collection system 102 or disturb the collection container 130. Disturbing the collection container 130 may cause retrograde fluid flow through the system towards the patient, causing discomfort or increasing the risk of infection. Advantageously, the UO device 150 can monitor fluid levels more frequently (e.g. 1000 times per minute), than would be possible or feasible by a clinician, providing much higher accuracy of fluid volume and flow data.

In an embodiment, the automatic fluid output system 100 including the UO device 150 and image processing device 170 can remotely monitor the fluid disposed within the collection container 130 for the presence/absence or concentration of foreign particles 80, or changes thereof over time. As shown in FIGS. 4C-4D, high resolution, high contrast SWIR images of the collection container 130 can indicate the presence or concentration of foreign particles 80. Repeated imaging can determine changes in concentration of foreign particle 80 over time. In an embodiment, these images can be analyzed locally by the UO device 150. In an embodiment, these images can be communicated with one or more remote computing devices 90 for analysis or for further analysis by artificial intelligence (A.I.), machine learning systems or the like. Exemplary remote computing devices 90 can include BD Kiestra™ Urine Culture systems, or the like. Further details and embodiments of which can be found in US 2019/0233873; US 2020/0283719; US 2020/0342604; U.S. Pat. Nos. 9,180,448; 10,294,508; 10,841,503; 11,319,575; 11,341,648; and WO 2020/127692, which are herein incorporated by reference in their entirety into this application.

Advantageously, the UO device 150 can determine the presence/absence or concentration of foreign particles 80 within the collection container 130 without having to directly sample the fluid or traverse the closed fluid collection system 102. This can maintain the integrity of the closed fluid collection system 102 and prevent the introduction of pathogens or the like. Advantageously, the closed fluid collection fluid collection system 102 including UO device 150 does not require any additional sampling ports, additional collection and measurement containers, weight based fluid measurement systems, or the like, simplifying manufacture and reducing associated costs.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An automatic fluid output measurement system, comprising:
a fluid output measurement device having a housing including a back plate supporting a short wave infrared (“SWIR”) camera, and a shield extending parallel to the back plate and supported in spaced apart relationship therefrom;
a fluid collection system including a collection container disposed between the back plate and the shield; and
an image processing device disposed within the housing and communicatively coupled to the SWIR camera, the image processing device configured to image the collection container using short wave infrared electromagnetic radiation to determine a volume of fluid disposed therein.

2. The automatic fluid output measurement system according to claim 1, further including a SWIR light source configured to emit short wave infrared electromagnetic radiation onto the collection container.

3. The automatic fluid output measurement system according to claim 1, further including a second camera communicatively coupled to the image processing device.

4. The automatic fluid output measurement system according to claim 3, wherein the second camera is disposed at one or both of a same tilt angle and a same focal length as the SWIR camera.

5. The automatic fluid output measurement system according to claim 3, wherein the second camera is disposed at one or both of a different tilt angle and a different focal length as the SWIR camera.

6. The automatic fluid output measurement system according to claim 1, wherein the SWIR camera is further configured to image the collection container using one or more of SWIR, medium wave infrared radiation (“MWIR”), long wave infrared radiation (“LWIR”), visible light, ultraviolet light, and infrared radiation (“IR”).

7. The automatic fluid output measurement system according to claim 1, wherein the collection container includes one or more graduated markings configured to provide a high contrast graduated marking relative to the collection container when imaged by the SWIR camera.

8. The automatic fluid output measurement system according to claim 1, wherein the collection container is configured to be suspended from the housing.

9. The automatic fluid output measurement system according to claim 1, wherein the housing includes a clamp configured to couple the housing to a supporting structure.

10. The automatic fluid output measurement system according to claim 1, wherein the image processing device is communicatively coupled to an external computing device.

11. The automatic fluid output measurement system according to claim 1, wherein the image processing device is configured to determine one or both of a presence of a foreign particle and a concentration of a foreign particle within the collection container.

12. The automatic fluid output measurement system according to claim 1, wherein the collection container is in fluid communication with a Foley catheter.

13. A method of measuring a fluid from a patient, comprising:
draining a fluid to a collection container;
imaging the collection container using a first camera configured to detect short wave infrared electromagnetic radiation; and
determining a fluid volume within the collection container.

14. The method according to claim 13, further including determining a change in fluid volume over time to determine a fluid flow.

15. The method according to claim 13, further including determining one or both of a presence of a foreign particle within the collection container and a concentration of a foreign particle within the collection container.

16. The method according to claim 13, further including detecting a first image using the first camera, and a second image using a second camera.

17. The method according to claim 16, wherein the first camera images one or both of a different portion of the collection container, a different angle of the collection container, and a different focal length, relative to the second camera.

18. The method according to claim 13, wherein the camera is supported by a housing having a back plate and a shield extending parallel to the back plate and supported in a spaced apart relationship therefrom, the collection container disposed between the back plate and the shield.

19. The method according to claim 13, further including suspending the housing from a supporting structure.

20. The method according to claim 13, further including imaging a graduated marking under short wave infrared electromagnetic radiation and comparing a fluid level relative to the graduated marking to determine a fluid volume.

21. The method according to claim 13, further including communicating the image of the collection container to an external computing device.

22. The method according to claim 13, wherein draining a fluid further includes draining urine from a catheter to the collection container.

23. The method according to claim 13, wherein the collection container includes one of a plastic, polymer, High Density Polyethylene (HDPE), Polyethylene Terephthalate (PET), Polypropylene (PP), Low Density Polyethylene (LDPE), or Polyvinyl Chloride (PVC).
